(19) United States
(12) Patent Application Publication
(10) Pub. No.: US 2016/0113516 A1
(43) Pub. Date: Apr. 28, 2016

(54) DENTAL DIAGNOSTIC DEVICE AND METHOD

(71) Applicant: Walter Robert CARNES, New Bern, NC (US)

(72) Inventor: Walter Robert CARNES, New Bern, NC (US)

(21) Appl. No.: 14/863,796

(22) Filed: Sep. 24, 2015

Related U.S. Application Data
(60) Provisional application No. 62/066,979, filed on Oct. 22, 2014.

(51) Int. Cl. A61B 5/01; A61D 5/00; A61B 5/00

(52) U.S. Cl. CPC A61B 5/015; A61B 5/4547; A61D 5/00; A61B 5/4552; A61B 5/742; A61B 2503/40

(57) ABSTRACT

A dental diagnostic device adapted to sense minute temperature variations along the gum and tooth root for detecting abscesses and infections therein, and a method of using the same is provided. The dental diagnostic device may include a handle portion joined to a sensor portion having a plurality of temperature sensors electronically connected to a computer with a user interface. The dental diagnostic device is dimensioned and adapted to allow an operator to sweep the sensor portion along a patient's upper and lower gum tissue, wherein the computer is adapted to electronically represent the output of the plurality of temperature sensors for detecting elevated local temperatures compared to surrounding tissue, indicating potential infection.

### CROSS-REFERENCE TO RELATED APPLICATION

This application claims the benefit of priority of U.S. provisional application No. 62/066,979, filed 22 Oct. 2014, the contents of which are herein incorporated by reference.

### BACKGROUND OF THE INVENTION

The present invention relates to the dental arts and, more particularly, to a low-cost dental diagnostic device adapted to sense minute temperature variations along the gum and tooth root for detecting abscesses and infections therein, and a method of using the same.

Currently, costly specialized roentgenography or X-ray photography is the primary clinical diagnostic tool in endodontic therapy and the dental arts for determining infection and/or inflammation of teeth and gum requiring root canal or related therapies. First, such methodologies may be unavailable, especially outside of a dental office and in lower-income areas, because of their prohibitive cost and lack of portability. Second, X-rays and their methodologies do not always provide evidence of latent infection because, for among other reasons, detection is limited since both healthy and necrotic pulps cast no image. As a result, current devices and methodologies are inaccurate in detecting abscesses and infected roots gums unless the infection is well advanced, possibly too advanced for some dental art therapies.

As can be seen, there is a need for a dental diagnostic device adapted to sense minute temperature variations along the gum and tooth root for detecting abscesses and infections therein, and a method of using the same.

### SUMMARY OF THE INVENTION

In one aspect of the present invention, a dental diagnostic device, includes a handle portion; and a plurality of temperature sensors joined to the handle portion, wherein the plurality of temperature sensors are dimensioned and adapted to operatively sweep along a gum line from a back of a jaw to a front of the jaw.

In another aspect of the present invention, a dental diagnostic device includes a handle portion; a sensor portion; and a plurality of temperature sensors disposed along the sensor portion, wherein the sensor portion is dimensioned to operatively sweep along a gum line from a back of a jaw to a front of the jaw so that the plurality of temperature sensors are operable to sense minute local temperature variations within gum tissue of the gum line, and wherein the jaw is at least one of a human, a canine, and a feline jaw.

In yet another aspect of the present invention, a computer-implemented method for detecting infections in a gum or tooth roots includes the steps of sweeping a plurality of temperature sensors along a gum line, wherein each temperature sensor is adapted to detect a plurality of temperatures within gum tissue of the gum line; establishing a dynamic temperature continuum of underling and local surrounding gum tissue along the gum line, wherein the dynamic temperature continuum is based on the plurality of temperatures; and further investigating a portion of the gum line associated with a predetermined temperature difference along the dynamic temperature continuum.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use; and

FIG. 2 is a perspective view of an exemplary embodiment of the present invention, shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a dental diagnostic device adapted to sense minute temperature variations along the gum and tooth root for detecting abscesses and infections therein, and a method of using the same. The dental diagnostic device may include a handle portion joined to a sensor portion having a plurality of temperature sensors electronically connected to a computer with a user interface. The dental diagnostic device is dimensioned and adapted to allow an operator to sweep the sensor portion along a patient's upper and lower gum tissue, wherein the computer is adapted to electronically represent the output of the plurality of temperature sensors for detecting elevated local temperatures compared to surrounding tissue, indicating potential infection.

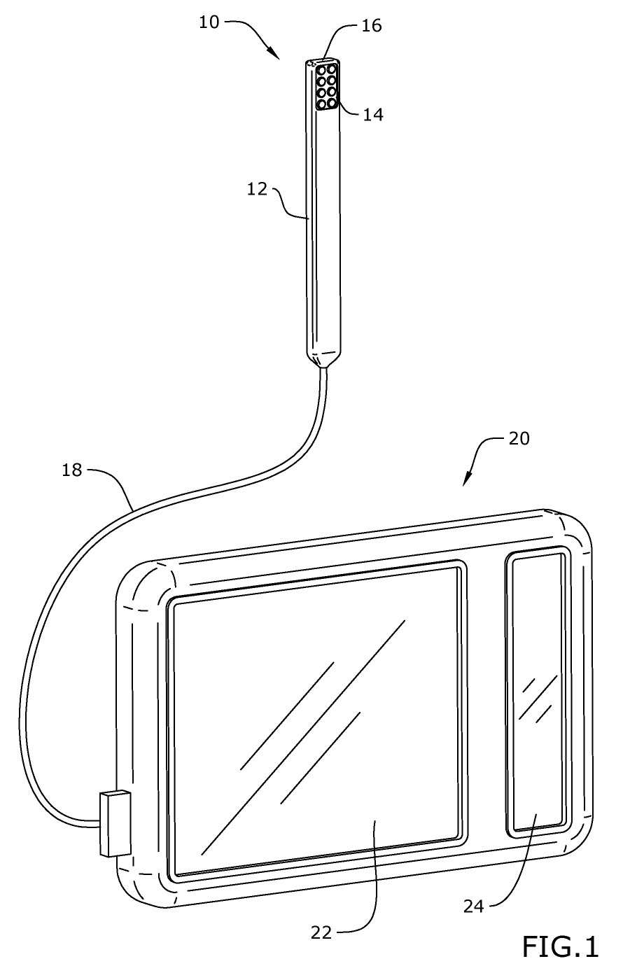

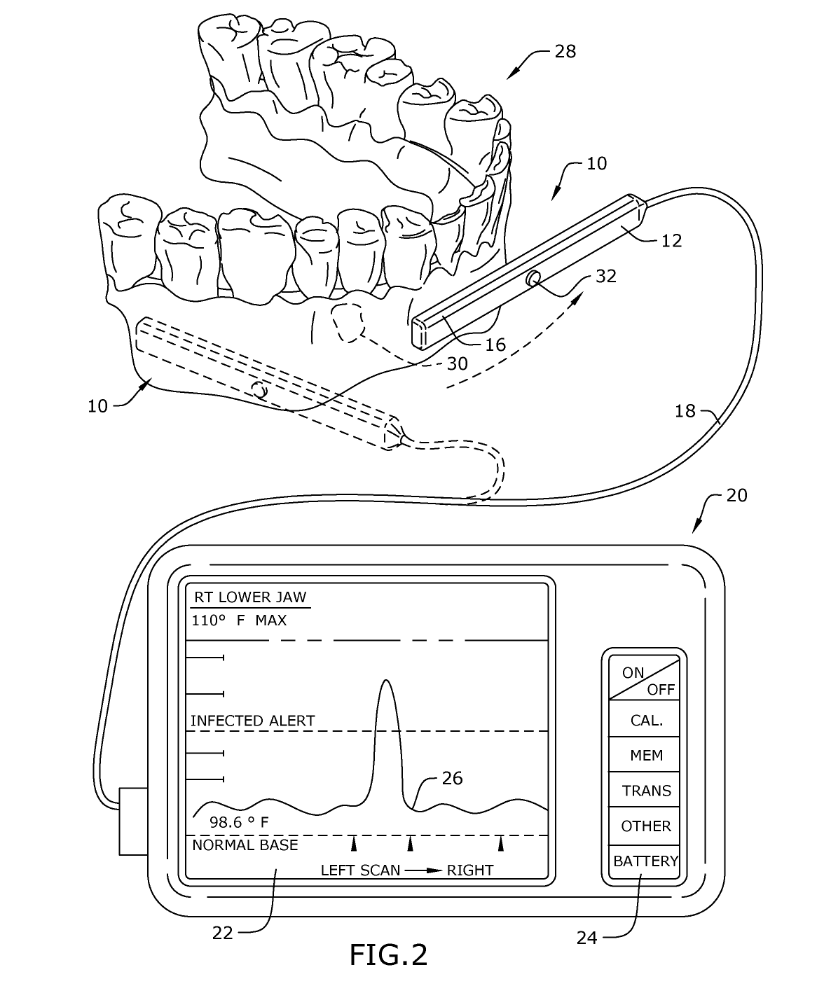

Referring to FIGS. 1 through 2, the present invention may include a dental diagnostic device 10 adapted to sense minute temperature variations along the gum and tooth root 28 for detecting abscesses and infections 30 therein, and a method of using the same. The dental diagnostic device 10 may include a handle portion 12 joined to a sensor portion 16. The handle portion 12 may be dimensioned and adapted to allow an operator to manually handle and otherwise manipulate the sensor portion 16. The sensor portion 16 may include a plurality of temperature sensors 14 adapted to sense minute, localized temperature variations in the gums and tooth roots The sensor portion 16 may be dimensioned so that the plurality temperature sensors 14 may be operable as the sensor portion 16 is ‘swept’ along the gum lines, from the back of a jaw to the front thereof. Accordingly, the sensor portion 16 may be adapted to slide between the gum line and the inner cheek of a patient. A push button 32 may be disposed along the handle portion 12, wherein the push button 32 is adapted to control power to the dental diagnostic device 10.

The plurality of temperature sensors 14 may be electronically connected to a computer 20 with a user interface 22, in certain embodiments remotely and, in alternative embodiments, by wiring 18. The computer 20 may include at least one processing unit electronically connected to a form of memory including, but not limited to, a desktop, laptop, and smart device, such as, a tablet and smart phone. The computer 20 includes a program product including a machine-readable program code for causing, when executed, the computer 20 to perform steps. The program product may include software which may either be loaded onto the computer 20 or accessed by the computer 20. The loaded software may include an application on a smart device. The software may be accessed by the computer 20 using a web browser. The computer 20 may access the software via the web browser using the internet, extranet, intranet, host server, internet cloud and the like.

The user interface 22 may be configured to produce electronic representations of a plurality of data sets 26 that are a function of the output of the plurality of temperature sensors 14 as well as the input of a user, in certain embodiments in the form of a read graph, as illustrated in FIG. 2. In certain embodiments, the computer may provide a control panel 24 for controlling parameters of the plurality of data sets 26 and their electronic representations.

The computer 20 may be adapted to be a power source for the dental diagnostic device 10. The computer 20 may be adapted to amplify and digitize the output signals of the plurality of temperature sensors 14. The computer 20 may be configured to process said plurality of data sets 26 so as to display the location of an abscess, infection and the like 30 along a portion of the gum and tooth roots, as illustrated in FIG. 2. In certain embodiments, the output of the plurality of temperature sensors 14 and/or plurality of data sets 26 may be represented relative to the entire set of teeth of the patient, through a dental tooth number chart, for example. The input of the user may include, but not be limited to, at least one base line temperature of a user's gum and root line temperatures. The computer 20 may be configured to adapt the plurality of data sets 26 so as to be displayed in graphical representations of fractional temperature variations between the output from the plurality of temperature sensors 14 and the at least one base line temperature. The computer 20 may be configured to electrically mark abnormal temperatures gradients relative to the at least one base line temperature.

A method of using the present invention may include the following. The dental diagnostic device 10 disclosed above may be provided. A user may manipulate the handle portion 12 so that the sensor portion 16 may be swept along a patient's upper and lower gum tissue from the back of the jaw to the front of the mouth, sensing and outputting a plurality of discrete temperatures (or data sets 26) along each gum line. The plurality of temperature sensors 14 may be adapted to be sensitive enough so that the output of the plurality of discrete temperatures may be used to determine minute temperature differentials along the swept gum tissue. The output of the plurality of discrete temperatures may be of such a nature that the determined differential temperatures of neighboring gum tissue can be analyzed for being indicative of potential infection around tooth root structure, such as root canal. The output of the plurality of temperature sensors 14 may be electronically transmitted to and processed by the computer 20, which in turn electronically represents the plurality of data sets 24 on the user interface 22, as illustrated in FIG. 2.

In certain embodiments, during the scanning procedure the dental diagnostic device 10 measures and displays a dynamic temperature continuum of the data sets 26 and/or the plurality of temperature sensors 14, wherein the dynamic temperature continuum includes a bright visual line from underling and local surrounding tissue and tooth/bone structure as the diagnostic device 10 is swept along the patient jaw/gum line and/or gum tissue. The scanning procedure automatically measures and records temperature in linear width increments of 2.0 millimeters during examinations the diagnostic device 10 is moved along gum line.

The graduated vertical temperature display scale range may be from a low 96.00 degrees Fahrenheit (change to Celsius measurement is switchable) to a maximum of 110.00 F. degrees F.

The vertical visual scale indicates temperature gradients—or steps—in one half degree increments Fahrenheit. Scanned tooth root or jaw gum line temperature and location exceeding a predetermined temperature difference is a cause for further investigation. Therefore, when the dynamic temperature continuum includes the predetermined temperature difference/differential further investigations is warranted.

As a result, portions of the gum tissue and/or tooth or bone structure associated said predetermined temperature difference/differential is investigated for detecting abscesses and infections therein. The predetermined temperature difference may be approximately 2.0 degrees Fahrenheit. In certain embodiments, the predetermined temperature difference would be a function of a normal body temperature, in excess thereof. The normal body temperature may be determined by the diagnostic device 10, for example, as a base line of the dynamic temperature continuum.

### Operation

Procedurally to achieve an accurate temperature analysis the dental diagnostic device 10 is initially briefly placed under the patient's tongue and records sets the patient body temperature threshold, typically 98.6 degrees F. If the body temperature is higher—indicating a body fever and or bodily infection the patient must be remanded for further examination.

Lower tissue temperature indicates lack of blood flow and potential diabetic or more extensive bodily conditions. The base threshold is recorded and logged into the devices patient history memory for later recall. The visual display recognizes the velocity of the scanning rate. The entire scanning procedure of the upper and lower jaw may be accomplished typically in less than a one minute.

In alternative embodiments, the dental diagnostic device 10 can detect abnormal fillings and voids in teeth and jaw bone of human and animals. The dental diagnostic device 10 might be used to detect micro flaws and cracks in hot turbine blades and accessories. In yet another embodiment, the dental diagnostic device 10 might be configured to detect hot electrical leakage in wire transmission.

The present invention is completely portable, rugged simple to operate and inexpensive compared to conventional Ray and radio-logical image detection methodology. The present invention requires little expertise to learn and determine dental infection problems in the field and can be used with minimal training for use in diagnostics of gum disease in both humans and animals.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A dental diagnostic device, comprising:
a handle portion; and
a plurality of temperature sensors joined to the handle portion,
wherein the plurality of temperature sensors are dimensioned and adapted to operatively sweep along a gum line from a back of a jaw to a front of the jaw.

2. The dental diagnostic device of claim 1, wherein the jaw is a human jaw.

3. The dental diagnostic device of claim 1, wherein the jaw is at least one of a canine or feline jaw.

4. The dental diagnostic device of claim 1, further comprising a sensor portion joined to the handle portion so that the plurality of temperature sensors are disposed on the sensor portion.

5. The dental diagnostic device of claim 1, wherein the plurality of temperature sensors are adapted to sense minute local temperature variations along the gum line.

6. A dental diagnostic device, comprising:
a handle portion;
a sensor portion; and
a plurality of temperature sensors disposed along the sensor portion,
wherein the sensor portion is dimensioned to operatively sweep along a gum line from a back of a jaw to a front of the jaw so that the plurality of temperature sensors are operable to sense minute local temperature variations within gum tissue of the gum line, and
wherein the jaw is at least one of a human, a canine, and a feline jaw.

7. A computer-implemented method for detecting infections in a gum or tooth roots, comprising:
sweeping a plurality of temperature sensors along a gum line, wherein each temperature sensor is adapted to detect a plurality of temperatures within gum tissue of the gum line;
establishing a dynamic temperature continuum of underling and local surrounding gum tissue along the gum line, wherein the dynamic temperature continuum is based on the plurality of temperatures; and
further investigating a portion of the gum line associated with a predetermined temperature difference along the dynamic temperature continuum.

8. The method of claim 7, wherein the predetermined temperature difference is between underlying and local surrounding gum tissue.

9. The method of claim 7, wherein the predetermined temperature difference is between underlying and local surrounding gum tissue and tooth or bone structure.

10. The method of claim 7, wherein the predetermined temperature difference approximately 2.0 degrees Fahrenheit.

11. The method of claim 7, further comprising setting up a base line temperature, and wherein the predetermined temperature difference is a function of the base line temperature.

12. The method of claim 7, further comprising electronically representing the dynamic temperature continuum so as to visually determine the predetermined temperature difference.

13. The method of claim 11, further comprising electronically representing the dynamic temperature continuum and the base line so as to visually determine the predetermined temperature difference.
